(19) United States
(12) Patent Application Publication
(10) Pub. No.: US 2025/0059383 A1
(43) Pub. Date: Feb. 20, 2025

(54) ANTIMICROBIAL SURFACE WITH CONDUCTIVE PARTICLES

(71) Applicant: 3M INNOVATIVE PROPERTIES COMPANY, St. Paul, MN (US)

(72) Inventors: Lijun Zu, Woodbury, MN (US); Minghua Dai, Plymouth, MN (US); Stephen A. O. Olson, Maplewood, MN (US); Ying Xia, Woodbury, MN (US); Qihong Nie, Woodbury, MN (US); Yen-Lin Wu, St. Paul, MN (US)

(21) Appl. No.: 18/721,254

(22) PCT Filed: Dec. 2, 2022
(86) PCT No.: PCT/IB2022/061713

§ 371 Date: Jun. 18, 2024

Related U.S. Application Data
(60) Provisional application No. 63/294,978, filed on Dec. 30, 2021.
(60) Provisional application No. 63/429,374, filed on Dec. 1, 2022.

(51) Int. Cl. C09D 5/14; A61L 2/18; A61L 101/26; C02F 1/461; C09D 7/62; C09D 167/02; C09J 7/35

(52) U.S. Cl. CPC C09D 5/14; A61L 2/18; C02F 1/46176; C09D 7/62; C09D 167/02; C09J 7/35; A61L 2101/26; A61L 2202/11; C02F 2303/04; C09J 2301/304; C09J 2467/00

(57) ABSTRACT

An antimicrobial article includes a supporting layer and an antimicrobial layer. The antimicrobial layer has a first major surface at least partially formed by a plurality of particles and a second major surface coupled to the supporting layer. Each particle of the plurality of particles is formed by a bead of nonconductive material. A first set of the particles include beads coated by a first conductive material.

### TECHNICAL FIELD

The present technology is generally related to antimicrobial surfaces and, in particular, to antimicrobial scratch-resistant surfaces.

### SUMMARY

In one aspect, the present disclosure relates to an antimicrobial article including a supporting layer and an antimicrobial layer. The antimicrobial layer has a first major surface at least partially formed by a plurality of particles and a second major surface coupled to the supporting layer. Each particle of the plurality of particles includes a bead of nonconductive material. A first set of the particles include beads coated by a first conductive material.

In another aspect, the present disclosure relates to a method of using the article. The method includes applying a liquid to the first major surface of the article.

The liquid is optionally an electrolytic solution.

### BRIEF DESCRIPTION OF DRAWINGS

FIG. 1 is a cross-section view of an antimicrobial article.

FIG. 2 is a cross-section view of the antimicrobial article of FIG. 1 after some use.

FIG. 3 is a plan view of an antimicrobial layer that may be used with the antimicrobial article of FIG. 1 having conductive particles and uncoated particles.

FIG. 4 is a plan view of another antimicrobial layer that may be used with the antimicrobial article of FIG. 1 having a higher ratio of conductive particles compared to uncoated particles than the antimicrobial layer of FIG. 3.

FIG. 5 is a cross-section view of an antimicrobial layer usable in the antimicrobial article of FIG. 1 having a plurality of barrier regions.

### DETAILED DESCRIPTION

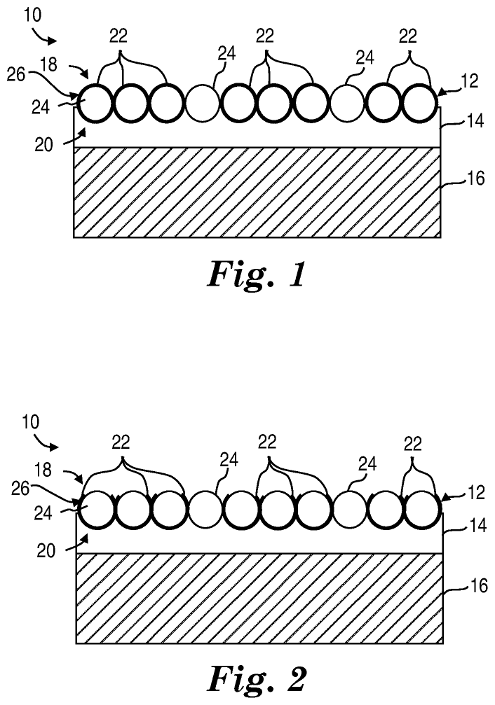

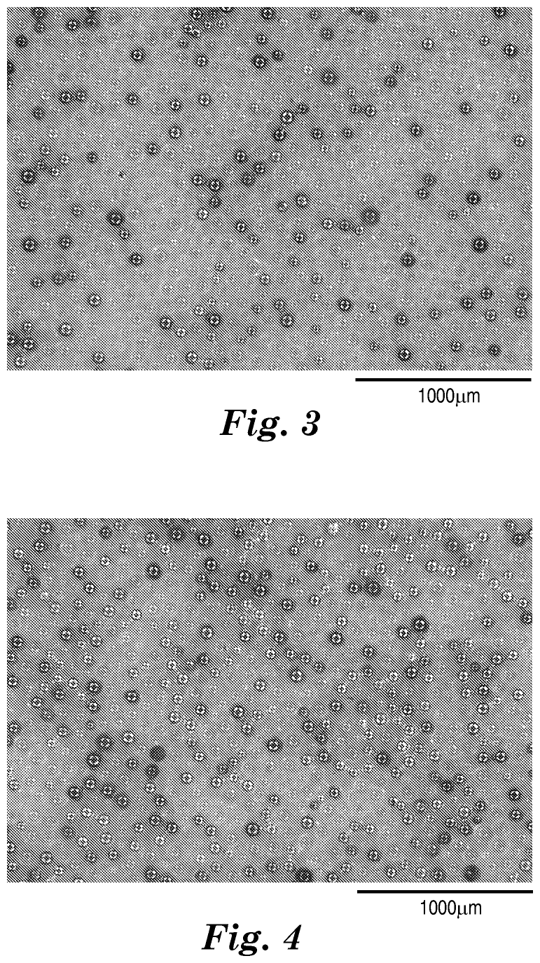

As can be seen in FIG. 1 and FIG. 2, the antimicrobial article 10 may be applied to, or be used with, a surface of a substrate 16 and may provide antimicrobial properties to the substrate surface, particularly antibacterial and antiviral properties. In general, the antimicrobial article 10 includes a supporting layer 14 and an antimicrobial layer 12 coupled to the supporting layer. Antimicrobial properties may be provided by various mechanisms, such as metal ion release or galvanic microcurrents. The article 10 may also provide a scratch-resistant surface, which may facilitate prolonged antimicrobial performance over a longer period of time compared to a substrate surface, such as a polymer film, without the antimicrobial article. The antimicrobial article 10 may be particularly useful, for example, as a durable, antimicrobial covering for high touch surfaces.

In terms of antimicrobial performance, the antimicrobial article 10 may provide a first major surface 18 capable of providing antibacterial, antiviral, or both types of performance at various minimum thresholds. Antimicrobial performance may be facilitated by the presence of moisture. Moisture may be provided by the ambient environment, such as moisture in the air. Moisture may also be applied, such as wiping with a rag wetted with water or an electrolytic solution.

Antimicrobial performance may be measured as a log reduction value (LRV). The log reduction value (LRV) is calculated according to Equation 1.

$$Equation 1$$

$$LRV = {log}_{10} [ \frac{\begin{matrix}concentration of organism recovered from a Control Sample \\ ( pfu / mL )\end{matrix}}{\begin{matrix}concentration of organism recovered from a Coated Sample \\ ( pfu / mL )\end{matrix}} ]$$

“Coated Sample” refers to the antimicrobial article 10 having at least the antimicrobial layer 12 on a supporting layer 14. “Control Sample” refers to a similar article without the antimicrobial layer 12, such as an article similar to article 10 including beads 24 except without a coating of conductive material 22. The organism may be a microbe, such as a bacteria or virus. A higher LRV indicates a greater reduction of organisms when a Coated Sample is compared to a Control Sample. LRV may be determined using measurements from a modified ISO 22196:2011 ‘Measurement of Antibacterial Activity on Plastic and Other Non-Porous Surfaces’ methods, which include the Bacteria Killing Test and Virus Killing Test, described herein elsewhere in more detail.

The antimicrobial article 10 may provide an LRV for an organism greater than or equal to 2, 3, 4, 4.5, or even 5 at 1 hour after being applied to the first major surface 18. In some embodiments, the article 10 may provide an LRV for bacteria greater than or equal to 2, 3, 4, 4.5, or even 5 at 1 hour after being applied to the first major surface 18. The article may even provide an LRV for bacteria greater than 5, for example, after 6 hours. In some embodiments, the article 10 may provide an LRV for a virus greater than or equal to 2, 3, 3.5, 4, or even 4.5 at 10 minutes after being applied to the first major surface 18.

As used herein, the term “major surface” means a largest, or one of the largest, surfaces of an object. For example, a film or paper article may have a first major surface, a second major surface, and side surfaces (or edges) connecting those major surfaces.

Various substrates 16 may be used with the antimicrobial article 10, which may be coupled to the supporting layer 14. In some embodiments, the substrate 16 may be a three-dimensional object, such as a table, countertop, door handle, steering wheel, chair armrest, appliance, electronic device, or other touchable or high-touch surfaces. In some embodiments, the substrate 16 may be a substantially planar article, such as a polymer film, polymer adhesive layer, fabric, paper, glass, wall material, or metal. Such substantially planar articles may be applied to a three-dimensional object. For example, the antimicrobial article 10 may be applied to a decorative film, which may then be applied to a tabletop. Although described as a separate component, the substrate 16 may also be considered part of the antimicrobial article 10.

The antimicrobial layer 12 includes a plurality of particles 26. The supporting layer 14 is configured to physically couple the particles 26 of the antimicrobial layer 12 into substantially fixed positions relative to one another along the article 10. As used herein, the term “couple” means to attach, bond, adhere, or otherwise physically link one component to another component.

Any suitable material capable of physically coupling, or binding, the plurality of particles 26 may be used to form the supporting layer 14. The supporting layer 14 may also be described as a binder or binder layer. In some embodiments, the supporting layer 14 is, or includes, a polymer adhesive or film. Non-limiting examples of material that may be used for the supporting layer 14 include polyolefin, polyester, polyurethane, vinyl, polyester, polyethylene, acrylic adhesives, epoxies, rubber, or silicone.

In one example, the antimicrobial article 10 includes the antimicrobial layer 12, a supporting layer 14 including polyester as a binder, and a substrate 16 including fabric, which may provide a flexible antimicrobial surface for clothing. In another example, the antimicrobial article 10 includes the antimicrobial layer 12 and a supporting layer 14 including a polymer adhesive, which may be applied to, or further include, a substrate 16 including a table.

The supporting layer 14 may have any suitable total thickness. The thickness may be greater than or equal to 10, 25, 50, 100, 250, or even 500 micrometers. The thickness may be less than or equal to 1000, 500, 250, 100, 50, or even 25 micrometers.

The antimicrobial layer 12 defines a first major surface 18 and a second major surface 20 opposite to the first major surface. The plurality of particles 26 of the antimicrobial layer 12 at least partially forms the first major surface 18. The plurality of particles 26 may also at least partially form the second major surface 20. The second major surface 20 is coupled to the supporting layer 14.

Each of the particles 26 includes a bead 24 of nonconductive material. As used herein, the term “bead” means a microsphere bead, which may be transparent, opaque, translucent, or colored. The beads 24 may be collectively described as having a mean diameter. The mean diameter may be greater than or equal to 10, 20, 30, 40, 50, 60, 70, 80, 90, or even 100 micrometers. The mean diameter may be less than or equal to 250, 200, 150, 100, 80, or even 60 micrometers. The size of the bead 24 may be selected, for example, to minimize the visual impact of the bead relative to the substrate.

Nonconductive material used to form the beads 24 may also be described as electrically insulating material. Any suitable nonconductive material may be used to form the beads 24. Examples of nonconductive materials include but are not limited to one or more of glass, ceramic, nonconductive metal oxide, cellulose or modified cellulose, and an electrically insulating polymer.

The nonconductive material may also be selected to provide a scratch-resistant major surface for the antimicrobial layer 12. In some embodiments, the nonconductive material may be selected to have a Mohs hardness greater than or equal to 4, 4.5, 5, 5.5, 6, 6.5, 7, 7.5, or even 8. For example, glass has a Mohs hardness of about 5.5.

The plurality of particles 26 may be distributed in any suitable manner to form the first major surface 18. In some embodiments, the plurality of particles 26 is formed as a monolayer, which may be described as a single layer of particles.

At least some of particles 26 include a coating of conductive material 22 and may be described as conductive particles. In particular, a first set of particles may include beads coated by a first conductive material. Any suitable conductive material 22 may be used suitable for coating the beads. The conductive material 22 may facilitate metal ion release or galvanic microcurrents. Non-limiting examples of material that may be used for the conductive coating material include Ag, Au, Pt, Pd, Ir, Cu, Sn, Sb, Bi, Zn, complexes, and colloids thereof. Metal oxides of the conductive coating material may also be used.

The conductive coating of conductive material 22 may have any suitable thickness. The thickness of the coating may be greater than or equal to 2, 10, 20, 50, 70, 100, or even 500 nanometers. The thickness of the coating may be less than or equal to 1000, 500, 100, 70, 50, 20, or even 10 nanometers. The conductive coating may be continuous or discontinuous on the beads 24 to at least partially form an outer, or exposed, surface of the particles 26. In some embodiments, a particular nonconductive material of the bead 24 and a particular conductive material 22 may be selected such that when a thin coating of the conductive material 22 is applied to the bead 24, a discontinuous coating may be provided that still provides antimicrobial properties. For example, coatings less than 10 micrometers, or even 20 micrometers, for some particles, may be discontinuously coated. The thickness of the coating may be selected to provide ion release and galvanic microcurrent properties to the antimicrobial article for a period suitable to the application of the article, such as a desired degree of metal ion release or galvanic microcurrent needed over a period of time. The coating may be applied to the bead in any suitable manner, including physical vapor deposition techniques, such as vacuum evaporation, sputtering, magnetron sputtering, and ion plating. Suitable physical vapor deposition techniques may include those described, for example, in U.S. Pat. Nos. 4,364,995; 5,681,575 and 5,753,251.

The coated particles can be fully coated or partially coated. For partially coated particles, at least some of the bead surface is exposed. In some embodiments, at least 5%, 10%, 20%, 30%, 50%, or 70% of the particle surface is exposed or uncoated. In some embodiments, no more than 95%, 90%, 80%, 70%, 60%, 50%, 40%, 30%, 20%, or 10% of the particle surface is exposed or uncoated.

More than one coating of conductive material 22 may be used. In some embodiments, a second set of the particles include beads coated by a second conductive material. The second conductive material may have a different electric potential than the first conductive material. For example, a first conductive material may include Ag or Cu and a second conductive material may include the other Cu or Ag.

The first set and the second set of particles 26 may be distributed in any suitable manner relative to one another throughout the antimicrobial layer, which may be a monolayer. In some embodiments, the first set and second set of particles 26 may be randomly distributed throughout the first major surface (see FIGS. 3 and 4).

In some embodiments, at least some of the particles 26 are uncoated by a conductive material and described as nonconductive particles. A third set of the particles may be uncoated by a conductive material. For example, some particles may include an uncoated and exposed bead 24. Any suitable amount of conductive particles relative to non-conductive, or uncoated, particles may be used. In general, the percentage of particles 26 including beads 24 coated by conductive material is greater than or equal to 5%, 10%, 15%, 20%, 25%, 40%, 50%, 75%, or even 100% of the total number of the plurality of particles.

The conductive and nonconductive particles 26 may be distributed in any suitable manner relative to one another throughout the antimicrobial layer, which may be a monolayer. In some embodiments, at least some of the conductive particles may be randomly distributed among the nonconductive particles, and vice versa (see FIGS. 3 and 4).

The antimicrobial article 10 may also be more efficient than existing techniques by concentrating conductive material at a contact surface, such as the first major surface 18. Some of the conductive material 24 may also be protected from being removed by scratching due to the contour of the beads 24. FIG. 2 shows the particles 26 after some of the coating of conductive material 22 has been worn off the beads 24. Conductive material 24 still remains in valleys between the beads 24 to facilitate release of ions or galvanic microcurrents.

FIG. 3 shows an antimicrobial layer 40 having a first set of coated particles, a second set of coated particles, and a third set of uncoated particles. FIG. 4 shows an antimicrobial layer 50 also having a first set of coated particles, a second set of coated particles, and a third set of uncoated particles. In the illustration, the coated particles have stronger and darker features, whereas uncoated particles appear relatively more faded with lighter gray features. However, antimicrobial layer 50 (FIG. 4) has a higher ratio of coated particles compared to uncoated particles than antimicrobial layer 40 (FIG. 3). In other words, antimicrobial layer 50 has a higher percentage of particles coated by conductive material than antimicrobial layer 40. In the illustrated embodiments, antimicrobial layer 40 has 80% of the uncoated particles, 10% of the first set of coated particles (Cu), and 10% of the second set of coated particles (Ag), whereas antimicrobial layer 50 has 50% of the uncoated particles, 25% of the first set of coated particles (Cu), and 25% of the second set of coated particles (Ag).

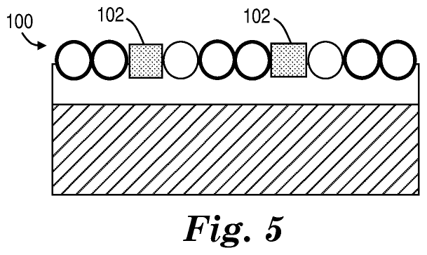

Barrier regions may be used to form the antimicrobial layer. FIG. 5 shows an antimicrobial layer 100 having barrier regions 102 disposed between particles. The barrier regions 102 may at least partially form the first major surface of the antimicrobial layer. The barrier regions 102 may also at least partially form part of the monolayer with the particles when the particles are arranged to form a monolayer.

The barrier region material may be applied in any suitable pattern or shape, including regular and irregular shapes, linear and curved shapes, continuous and discontinuous patterns, random and repeating patterns, and combinations thereof. The barrier regions 102 may be large or small in surface area. Pre-determined patterns of the barrier regions 102 and particles may be arranged to have a desired visual effect or so that patterns are not visible to the naked eye.

Barrier regions 102 may be formed using any suitable barrier region material. As used herein, the term “barrier region material” means a material that substantially prevents beads 24 (FIGS. 1 and 2) from attaching to a layer on which the barrier region material is applied, such as a polymeric supporting layer. Barrier region material may be permanent, quasi-permanent, or transient. Non-limiting examples of barrier materials include waxes, resins, polymeric materials, inks, inorganics, ultraviolet-curable (UV-curable) polymers, particles composed of either organic or inorganic metallic or non-metallic materials, or photoresist.

Any suitable amount of barrier regions 102 may be used. In some embodiments, the area of barrier regions 102 forming the first major surface may be greater than or equal to 5%, 10%, 15%, 20%, 25%, 40%, 50%, 75%, or even 90% the total surface area of the first major surface. Further examples of techniques and materials related to making and using the barrier region may be found in U.S. Pat. No. 10,845,514 (Chen-Ho et al.).

Antimicrobial articles may be made in any suitable manner. In one example, some beads may be coated with a thin layer of a first conductive material, other beads may be coated with a thin layer of a second conductive material, and further beads may remain uncoated. The beads may be selected from a same or similar size distribution (e.g., same mean diameter). The beads, before or after coating, may also be filtered to achieve a same or similar size distribution. The beads may be applied to a supporting layer, such as a polymer adhesive or film. The supporting layer, which may be an adhesive, may be applied to a substrate, such as a film or fabric.

Antimicrobial articles may provide antiviral or antibacterial properties via any suitable mechanism, which may include metal ion release or galvanic microcurrent effects upon applying a liquid, such as an electrolytic solution, to the first major surface. Applying a liquid may include the application of moisture from ambient air, which may be provided from the ambient environment. Metal ion release may be controlled, for example, using metal oxides and with application of an electrolytic solution, or fluid, to the first major surface. Galvanic microcurrent effects may be controlled, for example, by the number of conductive particles forming the first major surface, the electric potential between different coating materials, and by application of an electrolytic solution to the first major surface.

The ability to achieve release atoms, ions, molecules, or clusters of conductive material (e.g., metal) on a sustainable basis may be affected, for example, by varying the amount of the oxygen containing gas during vapor deposition. As the amount of metal oxide increases when the level of oxygen containing gas introduced increases, metal ions released from the article may in turn increase. Thus, a higher weight percent of metal oxide may, for example, provide an enhanced release of anti-microbial agents, such as metal ions and provide an increased antimicrobial activity.

When the first major surface is brought into contact with an alcohol or a water-based electrolyte, the conductive material may release ions, atoms, molecules, or clusters. The concentration of the conductive material may be selected sufficient to produce an anti-microbial effect based on the particular conductive material and the electrolytic solution.

The ability to generate at least one galvanic microcurrent (e.g., electrical current) when introduced to an electrolytic solution may affected by the conductive materials selected, particularly due to their difference in electric potential. Depending on the materials selected, a first conductive material may be a cathode (positive electrode) and a second conductive material may be an anode (negative electrode), when the electrons follow from the second conductive material to the first conductive material, and vice versa. Redox reactions and the flow of ions may take place in the presence of an electrolytic solution, and thus electrical currents may be produced between the first and second conductive materials. The current may be affected by the amount of conductive particles participating in the flow of ions. These currents can inhibit the growth of microbes, such as bacteria and virus.

Antimicrobial articles may be capable of generating an electrical current greater than or equal to 10, 50, 100, 250, 500, 1000, or even 2500 microamps (μA). when introduced to an electrolytic solution. The antimicrobial article may be capable of generating an electrical current less than or equal to 5000, 2500, 1000, 500, 250, 100, or even 50 HA when introduced to an electrolytic solution.

### EXAMPLES

Unless otherwise noted or readily apparent from the context, all parts, percentages, ratios, etc. in the Examples and the rest of the specification are by weight.

Materials

| Abbreviation | Description and Source |
|---|---|
| Sodium Chloride | Sodium Chloride - MilliporeSigma (St. Louis, MO) |
| tris-HCl | Tris(hydroxymethyl)aminomethane hydrochloride - |
| | MilliporeSigma (St. Louis, MO) |
| Magnesium chloride | Magnesium chloride - MilliporeSigma (St. Louis, MO) |
| PEG 8000 | Polyethylene glycol 8000 (average MW of 8000) - MilliporeSigma |
| | (St. Louis, MO) |
| LB Agar Powder | Luria Bertani agar powder - MilliporeSigma (St. Louis, MO) |
| LB Broth | LB Broth - MilliporeSigma (St. Louis, MO) |
| TSB | Tryptic Soy broth powder - MilliporeSigma (St. Louis, MO) |
| Tryptone | Tryptone - MilliporeSigma (St. Louis, MO) |
| Yeast extract | Yeast extract Y0875 - MilliporeSigma (St. Louis, MO) |
| Agar | Agar - MilliporeSigma (St. Louis, MO) |
| Polyester-based | A 50% solids polyester resin commercially available as “VITEL |
| adhesive | 3550 B” from Bostik Company (Wausatosa, WI) |
| Polyester fabric | A 100% polyester fabric 2.25 oz/sqyd (Milliken and Co.) |
| Stretchable hot melt | Bemis 3419, Bemis Associates Inc. |
| adhesive | |
| Bacteria Strains | Strains containing Cutibacterium acnes (ATCC 27352) - ATCC |
| | (Manassas, VA) and Escherichia coli |
| Phi6 phages | Enveloped Phi6 phages (DSM 21518) - DSMZ-German |
| | Collection of Microorganisms and Cell cultures GmbH |
| | (Braunschweig Germany) |
| Pseudomonas strain | Pseudomonas strain (DSM 21482) - DSMZ-German Collection of |
| | Microorganisms and Cell cultures GmbH (Braunschweig |
| | Germany) |
| Gram-positive strain | Staphylococcus aureus subsp. Rosenbach strain (ATCC 6538) - |
| | DSMZ-German Collection of Microorganisms and Cell cultures |
| | GmbH (Braunschweig Germany) |
| Gram-negative | Escherichia coli (Migula) Castellani and Chalmers strain (ATCC |
| strain | 25922) - DSMZ-German Collection of Microorganisms and Cell |
| | cultures GmbH (Braunschweig Germany) |
| PET film | polyethylene terephthalate film |

### Bacteria and Phage Material

Soft TSB Agar was prepared by adding 40-gram TSB powder and agar (7.5 g) to deionized water (1 L). The resulting product was autoclaved (121° C. for 15 minutes). Prior to use, the soft agar was melted using a microwave oven and 0.05 ml of 1×10⁸ Pseudomonas strain (DSM 21482) was added per plate.

LB agar plates were prepared by adding LB agar powder (35 g) to deionized water (1 L), autoclaving the mixture (121° C. for 15 minutes), and pouring 15 ml of the molten gel-mixture into a plate. LB broth was prepared by dissolving LB broth powder (20 g) in deionized water (1 L) and autoclaving the mixture (121° C. for 15 minutes). TSB broth was prepared by suspending Tryptic Soy broth powder (30 g) in deionized water (1 L) and autoclaving the mixture (121° C. for 15 minutes).

Phi 6 phages were harvested from a liquid TSB culture of its host Pseudomonas strain cultured in a shake flask at 25° C., 250 RPM shaker for at least 8 hours.

Bacteria Strains were inoculated into LB broth and cultured in tubes at 25° C., 250 RPM shaker for at least 12 hours.

Staphylococcus aureus subsp. Rosenbach strain (ATCC 6538) were inoculated into TSB broth and cultured at 25° C., 250 RPM shaker for at least 12 hours.

Escherichia coli (Migula) Castellani and Chalmers strain (ATCC 25922) were inoculated into TSB broth and cultured at 25° C., 250 RPM shaker for at least 12 hours.

### Glass Beads

Two types of metallic-coated glass beads were separately prepared, for the examples as shown in Table 1, Cu-coated glass beads and Ag-coated glass beads using the following method. Glass Beads were baked at 150 C for at least 8 hours and then placed in a vacuum chamber (commercially available from Sierra Applied Sciences) equipped with a 300-mL particle agitator and a 3-inch diameter metal sputtering target and cathode. During deposition, the particle agitator was operated at about 4 revolutions per minute (rpm). The chamber was pumped down to a base pressure and metal was sputtered at a pressure of about 4 millitorr (mTorr) using argon gas (99.999% purity). The argon flow rate was 100 standard cubic centimeter per minute (sccm). The metal was sputtered desired settings to create a thin film coating of metal on the surface of the beads. The coated beads were filtered a 120-mesh filter. The copper coating was estimated to comprise 2.06% of final weight of the Cu-coated Glass Beads. The silver coating was estimated to comprise 1.60% of final weight of the Ag-coated Glass Beads.

TABLE 1
Metallic-Coated Glass Bead Preparatory Example Details

| Preparatory | Uncoated Glass | Sputtering | Chamber Base | |
|---|---|---|---|---|
| Examples: | Beads (grams) | Target | Pressure (Torr) | Sputter Settings |
| Cu-coated | 750.85 | Copper | 6.4 × 10−6 | 2.0 kW for 1.9 hours |
| Ag-coated | 750.11 | Silver | 8.1 × 10−6 | 0.5 kW for 2 hours |

### Examples 1-10 and Comparative Example 1

Examples were prepared by the following and with glass bead quantities and an oven temperature in Table 2. The glass beads were between 60-100 micrometers in diameter. Coated and uncoated glass beads, according to Table 2, were mixed in a glass or metal beaker and heated in an oven until the glass bead mixture reached oven temperature. A thermocouple probe was used to measure the temperature of the glass bead mixture. The heated mixture was then cascaded on top of and sunk into the polyethylene layer of a bead carrier liner. The bead carrier liner was prepared as described in U.S. Pat. No. 5,474,827 where a 20-50 micrometer polyethylene layer was coated on a paper backing. The sink depth was smaller than the diameter of the glass beads. A portion of the glass bead mixture remained exposed above the surface of the polyethylene layer.

TABLE 2
Compositions and processing conditions of examples

| | Uncoated | Cu-Coated | Ag-Coated | Oven |
|---|---|---|---|---|
| | Glass Beads | Glass Beads | Glass Beads | Temperature |
| Examples | (grams) | (grams) | (grams) | (° C.) |
| Example 1 | 40 | 5 | 5 | 132 |
| Example 2 | 25 | 12.5 | 12.5 | 132 |
| Example 3 | 10 | 20 | 20 | 132 |
| Example 4 | 25 | 0 | 25 | 132 |
| Example 5 | 12.5 | 37.5 | 0 | 93 |
| Example 6 | 25 | 25 | 0 | 93 |
| Example 7 | 37.5 | 12.5 | 0 | 93 |
| Example 8 | 45 | 5 | 0 | 93 |
| Example 9 | 0 | 50 | 0 | 93 |
| Example 10 | 0 | 0 | 50 | 132 |
| Comparative | 50 | 0 | 0 | 93 |
| Example 1 | | | | |

### Example 11: Cu-Coated PET Film

Cu films were deposited on 10.16 cm by 10.16 cm square sheets of PET film. The PET sheets were primed on one side. Metal was deposited on the primed surface of the PET film. Deposition was performed using a PVD75 vacuum deposition chamber (Kurt J Lesker, Jefferson Hills, PA, USA). Cu films were deposited using RF Sputtering. The power supply was a Kurt J Lesker model R301 RF power supply. The chamber was pumped down to a base pressure equal to or lower than 3.3×10⁻⁶ Torr. Ultra-high purity Argon gas was flowed to the chamber at a rate of about 51 sccm. The run pressure was about 4.1×10⁻³ Torr. Cu was sputtered at a set point of 200 Watts. Reflected power was 2.0 Watts. The substrate was rotated at 20 rpm on the chamber platen. Deposition was performed for 715 seconds, resulting in an estimated coating thickness of about 100 nm.

### Example 12: Ag-Coated PET Film

Ag films were deposited on 10.16 cm by 10.16 cm square sheets of PET film. The PET sheets were primed on one side. Metal was deposited on the primed surface of the PET film. Deposition was performed using a PVD75 vacuum deposition chamber (Kurt J Lesker, Jefferson Hills, PA, USA). Ag films were deposited using DC Magnetron Sputtering. The chamber was pumped down to a base pressure equal to or lower than 5.0×10⁻⁶ Torr. Ultra-high purity Argon gas was flowed to the chamber at a rate of about 51 sccm. The run pressure was about 4.1×10⁻³ Torr. Ag was sputtered at a set point of 200 Watts (300 Volts, 0.67 Amps). The substrate was rotated at 20 rpm on the chamber platen. Deposition was performed for 170 seconds, resulting in an estimated coating thickness of about 100 nm.

### Example 13: Article with Fabric Backing

The bead coated liner (Example 9) was coated 8 mil wet thickness with a polyester-based adhesive (a 50% solids polyester resin commercially available as “VITEL 3550 B” from Bostik Company, Wausatosa, WI), followed by a thermal treatment for 30 seconds at 190F, laminated to a 100% polyester fabric (2.25 oz/sqyd, Milliken and Co.), and then thermal treatment for 6 min at 210F. Following the thermal treatment process, the carrier layer was stripped away, exposing the previously embedded surfaces of the monolayer of glass microspheres to produce an article with fabric backing.

### Example 14: Article with Stretchable Hot Melt Adhesive Backing

The bead coated liner (Example 9) was coated 8 mil wet thickness with a polyester-based adhesive (a 50% solids polyester resin commercially available as “VITEL 3550 B” from Bostik Company, Wausatosa, WI), followed by a thermal treatment for 30 seconds at 190F, and for 6 min at 210F. After thermal treatment, the exposed surface of the coated adhesive layer was laminated to a stretchable hot melt adhesive (Bemis 3419, Bemis Associates Inc.). Following the lamination process, the carrier layer was stripped away, producing a co-stretchable article with the previously embedded surfaces of the monolayer of glass beads exposed.

### Measurement of Micro-Current

Examples 1-3 were cut into 2.54 cm by 5.08 cm sample coupons. Sample coupons were pre-wetted with Eyesaline (Honeywell, Charlotte, North Carolina). The micro-current was then measured using OTII-ARC-001 multi-meter with two-point probes (Scheelevägen, Sweden), shown in Table 3.

TABLE 3
Micro-current data for Examples 1-3

| Examples | Average Micro-current (μA) |
|---|---|
| Example 1 | 230 |
| Example 2 | 310 |
| Example 3 | 560 |

### Bacteria Killing Test

Modified ISO 22196:2011 method “Measurement of Antibacterial Activity on Plastic and Other Non-Porous Surfaces’ was used for evaluating the antibacterial properties of articles of the Examples. “Bacteria Killing Test” refers to this method described herein. Examples 1-12 were cut into circular coupons 2.54 cm diameter, n=3. A 25-microliter inoculum of bacteria, such as Bacteria Strains, gram-positive strain or gram native strain, was prepared in phosphate buffer with a concentration of 1×10E8-1×10E9 colony forming units/mL (cfu/mL) to be used for the tests.

The coupons were put in contact with bacteria by sandwiching the inoculum between the test material and a sterile microscope slide cover and incubated for 1 and/or 6 hours at room temperature. A coupon of Comparative Example 1 with a non-antimicrobial surface was treated with the inoculum. After incubation, coupon samples were neutralized in Dey\Engley neutralizing broth neutralizing broth (obtained from Becton Dickinson Company, Franklin Lakes, NJ) and accessed for viable cell count using a plate count culture method. For the plate count culture method, viable bacteria were enumerated by performing 10-fold serial dilutions. An aliquot of each dilution was spread-plated onto a LB plate or 3M™ PETRIFILM™ Rapid Coliform Count Plates. The plates were incubated for 16 hours at 37° C. Colonies on the LB plates were manually counted and reported in Table 4. Colonies on the Rapid Coliform Count Plates were counted with 3M™ PETRIFILM™ Plate Reader Advanced and reported in Table 5.

### Virus Killing Test

The virus killing test for antiviral activity was conducted using bacteria phages as surrogates. Modified ISO 22196:2011 method “Measurement of Antibacterial Activity on Plastic and Other Non-Porous Surfaces’ was used for evaluating the antivirus properties of articles of the Examples. “Virus killing test” refers to this method described herein. Examples 1-8 were cut into circular coupons 2.54 cm diameter, n=3. A 25-microliter Phi 6 phage solution with 10E10 pfu/ml was used for the assay.

The coupons were put in contact with virus by sandwiching the virus between the test material and a sterile microscope slide cover and incubated for 10 min at room temperature. A coupon of Comparative Example 1 with a non-antimicrobial surface was treated with the inoculum. After incubation, coupon samples were neutralized in 10 ml of Dey\Engley neutralizing broth neutralizing broth and accessed for viable Phi 6 phage count using a plate count culture method. For the plate count culture method, viable Phi 6 phage were enumerated by performing 10-fold serial dilutions. An aliquot of each dilution (100 microliters) was spread-plated onto a TSB soft agar plate with Pseudomonas strain (DSM 21482). The plates were incubated for at least 8 hours at room temperature. Phage plaques were manually counted and reported in Table 4.

TABLE 4
Bacteria Killing and Virus Killing Test Results: Log
Reduction Value of Bacteria Strains and Phi 6 Phage

Log Reduction Value (LRV)

| | Bacteria Killing | Virus Killing |
|---|---|---|
| Examples | in 6 hours | in 10 minutes |
| Example 1 | >5 | 4.66 |
| Example 2 | >5 | 4.66 |
| Example 3 | >5 | 4.66 |
| Example 4 | >5 | 0.70 |
| Example 5 | >5 | 4.66 |
| Example 6 | >5 | 4.66 |
| Example 7 | >5 | 4.66 |
| Example 8 | >5 | 4.17 |

TABLE 5
Bacteria Killing Test Results: Log Reduction
Value of Gram-positive and negative strains

| | Efficacy to Gram-positive | | Efficacy to Gram-negative | |
|---|---|---|---|---|
| | Log Reduction | Log Reduction | Log Reduction | Log Reduction |
| | Value (LRV) in | Value (LRV) in | Value (LRV) in | Value (LRV) in |
| Examples | 1 hour | 6 hour | 1 hour | 6 hour |
| Example 2 | 2.48 | 3.41 | 4.14 | 4.22 |
| Example 4 | 0.63 | −0.47 | −0.30 | 0.20 |
| Example 5 | 2.01 | 3.90 | 4.38 | 4.22 |
| Example 8 | 1.19 | 2.43 | 1.71 | 4.22 |

### Scratch and Bacteria Killing Test

Examples 9-12 were individually tested for scratch resistance using a TABER Model 5750 Linear Abrader (Taber Industries, North Tonawanda, NY). Firstly, a 2.5 cm by 5.0 cm section of a SCOTCH-BRITE™ hand pad 7447 (3M Company, St. Paul, MN) was adhesively attached to the bottom of the instrument testing arm and used as the abrasive material in the test. Each Example (10 cm by 10 cm) was adhesively attached to a horizontally positioned glass surface with the metal-coated surface exposed for contact with the abrasive pad. In operation, the abrasive pad was placed in contact with the metal-coated surface and operated in a linear back and forth motion across the metal-coated surface for 50 cycles (frequency of 60 cycles/minute) with a load of 825 grams attached to the upper end of the testing arm. Then rotate each Example by 90 degree and scratched for another 50 cycles under the same condition. Secondly, a 2.5 cm by 5.0 cm section of a SCOTCH-BRITE™ hand pad 7448 (3M Company, St. Paul, MN) was attached to the bottom of the instrument testing arm and scratched the same area of each Example for 100 cycles at 0 degree and 90 degrees, respectively. Thirdly, a 2.5 cm by 5.0 cm section of 3M TRIZACT™ HOOKIT™ Finishing Foam Disc, 3000 (3M Company, St. Paul, MN) was attached to the bottom of the instrument testing arm and scratched the same area of each Example for 100 cycles at 0 degree and 90 degrees, respectively.

The resulting scratched examples 9-12 were then tested for bacteria killing with Bacteria Strains and Gram-negative strain following the Bacteria Killing Test described above. The results were reported in Table 6 and Table 7.

TABLE 6
Bacteria Killing results for Examples 9-12
with Bacteria Strains after scratch test.

Log Reduction Value (LRV)

| | Bacteria Killing | Bacteria Killing |
|---|---|---|
| Examples | in 1 hour | in 6 hours |
| Example 9 | 5 | 5 |
| Example 10 | 2.5 | 5 |
| Example 11 | 1.2 | 5 |
| Example 12 | 0.3 | 2.2 |

TABLE 7
Bacteria Killing results for Examples 9-12 with
Gram negative strain after scratch test.

Log Reduction Value (LRV)

| | Bacteria Killing | Bacteria Killing |
|---|---|---|
| Examples | in 1 hour | in 6 hours |
| Example 9 | 4.82 | 4.62 |
| Example 10 | 0.08 | 4.22 |
| Example 11 | 4.93 | 4.62 |
| Example 12 | 0.51 | 2.46 |

Thus, various embodiments of ANTIMICROBIAL SURFACE WITH CONDUCTIVE PARTICLES are disclosed. Although reference is made herein to the accompanying set of drawings that form part of this disclosure, one of at least ordinary skill in the art will appreciate that various adaptations and modifications of the embodiments described herein are within, or do not depart from, the scope of this disclosure. For example, aspects of the embodiments described herein may be combined in a variety of ways with each other. Therefore, it is to be understood that, within the scope of the appended claims, the claimed invention may be practiced other than as explicitly described herein.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The term “or” is generally employed in its inclusive sense, for example, to mean “and/or” unless the context clearly dictates otherwise. The term “and/or” means one or all of the listed elements or a combination of at least two of the listed elements.

1. An antimicrobial article comprising:
a supporting layer; and
an antimicrobial layer having a first major surface at least partially formed by a plurality of particles and a second major surface coupled to the supporting layer, each particle of the plurality of particles comprising a bead of nonconductive material, wherein a first set of the particles include beads coated by a first conductive material.

2. The article of claim 1, wherein the first major surface is configured to provide at least one of:
a log reduction value for bacteria greater than or equal to 2 at 1 hour after being applied to the first major surface; and
a log reduction value for virus greater than or equal to 2 at 10 minutes after being applied to the first major surface.

3. The article of claim 1, wherein a second set of the particles include beads coated by a second conductive material having a different electric potential than the first conductive material.

4. The article of claim 3, wherein a current of at least 10 microamps is generated when the first major surface is coated with an electrolytic solution.

5. The article of claim 3, wherein the first conductive material comprises Ag and the second conductive material comprises Cu.

6. The article of claim 1, wherein the first conductive material comprises Cu.

7. The article of claim 1, wherein the plurality of particles is arranged to form a monolayer.

8. The article of claim 1, wherein a third set of the particles are uncoated by a conductive material.

9. The article of claim 8, wherein the particles having beads coated by conductive material are randomly distributed among the third set of particles.

10. The article of claim 1, wherein the first major surface is at least partially formed by a plurality of barrier regions disposed between the particles.

11. The article of claim 1, wherein the percentage of particles including beads coated by conductive material is greater than or equal to 5% of the total number of the plurality of particles.

12. The article of claim 1, wherein the nonconductive material of the beads has a Mohs hardness greater than or equal to 4.

13. The article of claim 1, wherein the nonconductive material of the beads comprise glass or ceramic.

14. The article of claim 1, wherein the supporting layer comprises a polymer adhesive or film.

15. A method of using the article of claim 1 comprising applying a liquid to the first major surface of the article, wherein the liquid is optionally an electrolytic solution.
